(19) United States
(12) Patent Application Publication
(10) Pub. No.: US 2024/0112795 A1
(43) Pub. Date: Apr. 4, 2024

(54) SYSTEMS AND METHODS TO SELECT AND SHARE LEARNING CONTENT BASED ON SIMILAR ACTIVITIES

(71) Applicant: KONINKLIJKE PHILIPS N.V., EINDHOVEN (NL)

(72) Inventors: Sri Andari HUSEN, EINDHOVEN (NL); Paul Anthony SHRUBSOLE, ARNHEM (NL); Frank BERGNER, HAMBURG (DE)

(21) Appl. No.: 18/369,983

(22) Filed: Sep. 19, 2023

Related U.S. Application Data
(60) Provisional application No. 63/411,168, filed on Sep. 29, 2022.

(51) Int. Cl. G16H 40/40; G06F 21/62; G06F 40/117; G06F 40/169; G06Q 10/20

(52) U.S. Cl. CPC G16H 40/40; G06F 21/6245; G06F 40/117; G06F 40/169; G06Q 10/20

(57) ABSTRACT

At least one database stores reference content. In an annotation method, an annotation to an item of the reference content is received via a user interface, an annotation context of the annotation is determined at least based on analysis of content of the annotation, and the item of the reference content is updated in the database with the annotation tagged with the annotation context. In a reference content presentation method, an item of the reference content is presented via the user interface, a presentation context of the presenting is determined, and it is determined whether a comparison of the annotation context and the presentation context satisfies a sharing filter. In response to the sharing filter being satisfied, the annotation is presented via the user interface. The annotation is not presented if the sharing filter is not satisfied.

### CROSS REFERENCE TO RELATED APPLICATIONS

This application claims the benefit of U.S. Provisional Patent Application No. 63/411,168 filed Sep. 29, 2022. This application is hereby incorporated by reference herein.

### FIELD

The following relates generally to the medical arts, medical education arts, and medical device operations educational content tracking arts, especially as directed to medical imaging devices.

### BACKGROUND

Complex medical devices offer great flexibility in how they can be used to diagnose, monitor, or treat patients. The performance of the medical device may depend strongly on how the operator uses the device, e.g. setting a non-optimal configuration may provide sub-optimal results whereas using a more optimal configuration may provide better results. Moreover, medical devices that are connected to the Internet or another electronic network may receive software or firmware upgrades over the network that provide new features or enhance existing features; however, these may be useless if the operator is not trained to effectively use the new or enhanced features. Thus, there is substantial benefit to offering education and support to get the best results from the medical devices according to the clinical needs of patients and according to the specializations, way of working of the staff, and the type of hospital or clinical practice.

Health care professionals need to be prepared for unfamiliar situations they might be presented with during their daily practice. To do this, they need to learn how to conduct specific procedures, workflows, protocols, or practices for certain clinical cases/patient-situations that are presented to them. Since such highly specific situations or cases occur infrequently, the training and education that they receive is not as effective as it could be because the specific knowledge that was gained about how to deal with such cases will fade over time.

Many professionals would like to share their experiences or useful content with others in the profession, especially when such content has the utility to solve a specific problem that others in their profession might experience in the future. For example, clinical staff or maintenance staff might find a use of a piece of technical content that helped them solve a problem that others are not aware of. However, the sharing of such content (annotated learning content, specific instructional content, protocol guidelines, best practices, imaging guidelines) within a team or across sites (such as hospitals) is typically done ad-hoc and is often not applicable to all professionals at a particular moment in time.

The following discloses certain improvements to overcome these problems and others.

### SUMMARY

In one aspect, a non-transitory computer readable medium stores at least one database storing reference content, and instructions readable and executable by at least one electronic processor to perform an annotation method. The annotation method includes: receiving an annotation to an item of the reference content via a user interface (UI); determining an annotation context of the annotation at least based on analysis of content of the annotation; and updating the item of the reference content in the database with the annotation tagged with the annotation context.

In another aspect, a method is disclosed of presenting reference content stored in a database. The database further stores annotations to the reference content tagged with annotation contexts. The method includes: presenting reference content from the database to a current user via a user interface (UI); matching a context of the current user to the annotation contexts of the stored annotations; and presenting each annotation whose annotation context matches the context of the current user while not presenting any annotation whose annotation context does not match the context of the current user.

In another aspect, a non-transitory computer readable medium stores: at least one database storing reference content; instructions readable and executable by at least one electronic processor to perform an annotation method; and instructions readable and executable by the at least one electronic processor to perform a reference content presentation method. The annotation method includes: receiving an annotation to an item of the reference content via a user interface (UI); determining an annotation context of the annotation at least based on analysis of content of the annotation; and updating the item of the reference content in the database with the annotation tagged with the annotation context. The reference content presentation method includes: presenting the item of the reference content via the UI; determining a presentation context of the presenting; determining whether a comparison of the annotation context and the presentation context satisfies a sharing filter; and, in response to the sharing filter being satisfied, presenting the annotation via the UI. The annotation is not presented if the sharing filter is not satisfied.

One advantage resides in providing targeted sharing of information amongst healthcare professionals.

Another advantage resides in tailoring shared information having a high relevance for similar healthcare professionals.

Another advantage resides in providing a mechanism for healthcare professionals to share information in the field with other professionals at a similar level.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative clinical content management system in accordance with the present disclosure.

FIGS. 2 and 3 show exemplary flow chart operations of the system of FIG. 1.

### DETAILED DESCRIPTION

A clinical content management system (clinical CMS) or knowledge management system can comprise a database of articles, tutorials, checklists, or other reference content for use by clinicians when performing medical procedures. There may be an analogous CMS for use by biomedical engineers (biomeds) or other maintenance personnel in maintaining hardware and software of such equipment.

In some embodiments disclosed herein, an approach for targeted annotation of content is provided. An annotation user interface (UI) is provided in the CMS, via which a user can add comments or other annotations to content, optionally including links to corresponding medical images, links to other content in the CMS or in other databases or on the Internet, or other such documentation. While such annotations can be useful, providing this capability can easily produce a long list of annotations that can be difficult for users to navigate.

In practice it is expected that the content provided in the CMS will be generally applicable to most situations, and so many annotations will relate to “outliers” that are specific to a particular context. For example, a user may add an annotation about a change to a documented stenting procedure which however only applies in a peculiar clinical situation. Hence, a user in problem-solving mode who is navigating the long list of annotations will find most of the annotations are unrelated to the current problem being solved.

In view of the foregoing, a disclosed annotation system is context-driven. A context engine analyzes the annotation along with any other information available to extract an annotation context for the annotation. For example, if the annotation expressly describes the context this can be extracted. However, additionally the context engine may access other sources of contextual information, such as a location of the terminal or other device via which the user is entering the annotation matched in space and concurrent in time to a laboratory where the procedure is being performed, matching the annotation to an open or recently closed ticket of a medical device maintenance ticketing system, based on information in the annotation and information in the ticket, information obtainable by matching the annotation to a specific patient, et cetera.

The annotation UI may also provide for entry of a scope of sharing intended by the annotator, e.g., “share to all,” “share only to others with similar problem,” “share only within my hospital,” “share only within my department,” etc. This information can then serve as a further source of contextual information that is used to limit the sharing scope to that specified. Additionally, de-identification or obfuscation of patient-identifying information (PH), hospital-identifying information, or so forth may also be performed on the annotation based on the scope. The user-chosen scope of the annotation may also constitute additional information fed to the context engine, e.g. if the user limits the scope to the catheterization laboratory, then the scope is suitably limited to catheterization procedures.

To provide targeted dissemination of the annotations, in a corresponding presentation method in which the UI presents an item of the reference content, a sharing filter is employed, which limits sharing of the annotation to the context including the user-specified scope of sharing (if provided) and to the context for the annotation determined by the context engine. To this end, the sharing filter may leverage the context engine to determine the context of a current user in problem-solving mode who is using the presentation method, so as to match the context of the current user to the context of the available annotations and to share only those annotations whose context matches the context of the current user.

In some variant embodiments, the CMS could also proactively push annotations to targeted users based on the context. For example, in a biomed CMS that is linked to a help request ticketing system, the context of an annotation could be matched to contexts of open tickets in the ticketing system, and the annotation can be pushed to the owner of any ticket whose context matches the context of the annotation. A similar approach could be employed in a clinical CMS context based on access to a scheduler providing information on upcoming clinical procedures—an annotation can be pushed to the clinician scheduled to perform a procedure whose context matches the context of the annotation.

In another variant embodiment, the targeted annotation system could be useful in providing synergistic cross-over between the biomed CMS and clinical CMS. For example, if an annotation to content in the clinical CMS has context that suggests it may relate to a technical problem with the equipment (e.g., the annotation indicates the user determined the apparent clinical problem was actually an equipment malfunction), then this annotation could be pushed to users of both the biomed CMS and clinical CMS based on context. Similarly, if an annotation to content of the biomed CMS indicates that the observed problem is actually not due to an equipment malfunction but rather to an unusual presentation of a particular patient that is properly detected by the equipment, then this annotation can also be pushed to users of both the biomed CMS and clinical CMS based on context.

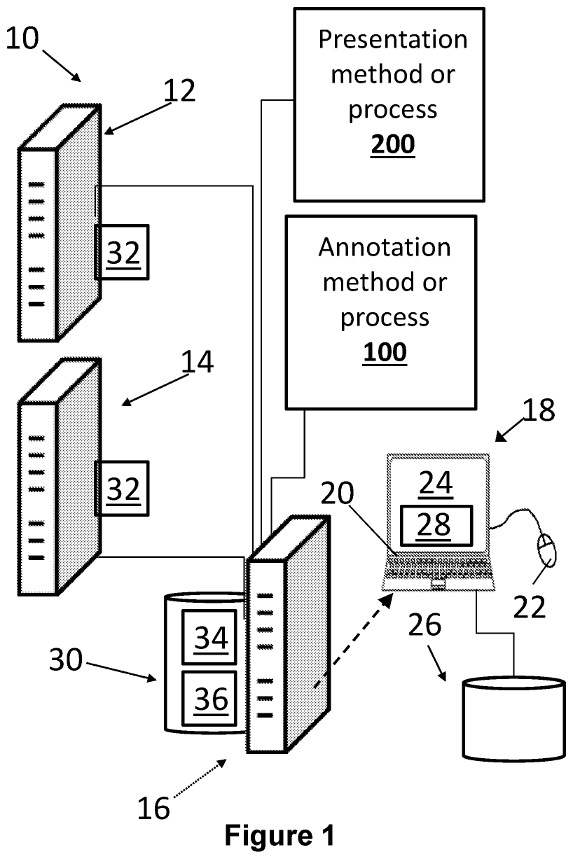

With reference to FIG. 1, an illustrative clinical content management system or apparatus 10 for providing annotations for reference content for one or more medical procedures. The apparatus 10 includes, or is in communication with, a clinical CMS 12 and a biomed CMS 14. The clinical CMS 12 comprise a database of articles, tutorials, checklists, or other reference content for use by clinicians when performing medical procedures. The biomed CMS 14 is used by bio-meds or other maintenance personnel in maintaining hardware and software of equipment used in the medical procedures. The clinical CMS 12 and/or the biomed CMS 14 stores reference content 32 comprising one or more of articles, tutorials, or checklists for use by clinicians or biomeds when performing medical procedures using a medical device. The clinicians and biomeds can access the reference content 32 during, before, or after a medical procedure. As shown in FIG. 1, the clinical content management system 10 includes, or is accessible by, a server computer 16 typically disposed remotely from the clinical CMS 12 and/or the biomed CMS 14 for which the reference content 32 is provided.

FIG. 1 also shows an electronic processing device 18, such as a workstation computer, a tablet, or more generally a computer. Additionally or alternatively, the electronic processing device 18 can be embodied as a server computer or a plurality of server computers, e.g. interconnected to form a server cluster, cloud computing resource, or so forth. The electronic processing device 18 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the workstation 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the display device 24.

The server computer 16 comprises a computer or other programmable electronic device that includes a non-transitory computer readable medium comprising a database 30 storing a set of annotations 34 for a user (i.e., a clinician or biomed) to annotate the reference content 32 with the annotations 34. The annotations 34 can include comments, links to medical images, links to other content, external documentation, and so forth.

The database 30 stores instructions executable by the server computer 16 to perform an annotation method or process 100 implemented by the system 10 for annotating the reference content 32 with the annotations 34. The database 30 also stores instructions executable by the server computer 16 to perform a presentation method or process 200 implemented by the system 10 for presenting reference content 32 with the annotations 34 with a sharing filter 36. In some examples, the method 100 and/or 200 may be performed at least in part by cloud processing (that is, the server computer 16 may be implemented as a cloud computing resource comprising an ad hoc network of server computers).

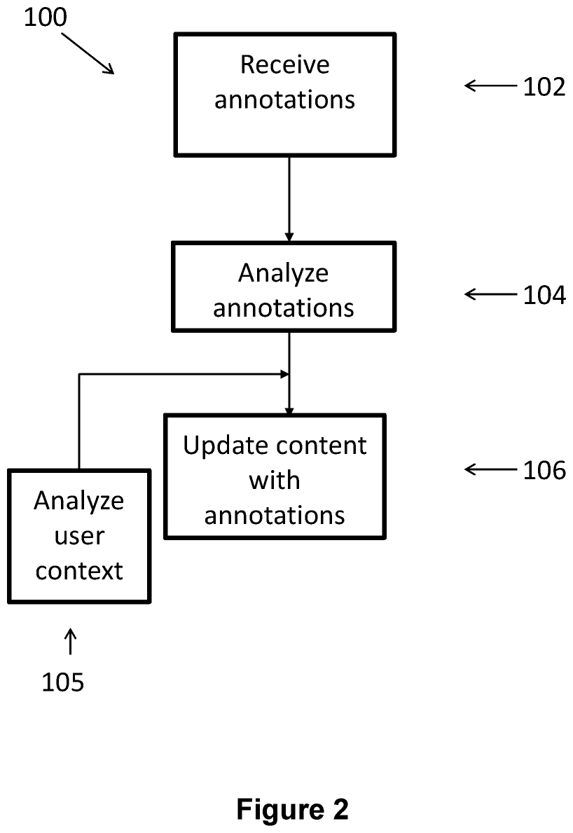

With reference to FIG. 2, and with continuing reference to FIG. 1, an illustrative embodiment of an instance of the method 100 is diagrammatically shown as a flowchart. To begin the method 100, the GUI 28 is provided on the display device 24 of the electronic processing device 18. At an operation 102, an annotation 34 to an item of the reference content 32 is received via the GUI 28 (e.g., using the at least one user input device 22).

At an operation 104, an annotation context of the annotation 34 is determined at least based on analysis of content of the annotation. In some embodiments, the analysis operation 104 includes analyzing additional data of contextual information. Such additional data of contextual information can include, for example, a location of the electronic processing device 18 matched in space and concurrent in time to a location where a medical procedure is being performed, matching the annotation 34 to an open or recently closed ticket of a medical device maintenance ticketing system, information obtainable by matching the annotation to a specific patient, and so forth. The reference content 32 can be updated with the annotations 34 when the context of the annotations 34 matches the context of the additional data.

In some embodiments, the operation 106 can include providing inputs indicative of a scope of sharing of the updated reference content 32 (e.g., “share to all”, “share only to others with similar problem”, “share only within my hospital”, “share only within my department”, etc.) The reference content 32 can then be updated with the input scope of sharing. In some embodiments, the scope of sharing of the updated reference content 32 can be altered with a filter 36 implemented in the server computer 16. The altering process can include matching a context of the current user to the context of the available annotations 34 whose context matches the context of the current user (e.g., clinician vs. biomed). The reference content 32 can then be updated with the altered scope of sharing.

In some embodiments, the operation 104 can include providing inputs indicative of removal of personally identifiable information (PII) of persons referenced in the annotations 34. The reference content 32 can then be updated with the PII removed.

In some embodiments, at an optional operation 105, a context of the current user selecting the annotations 34 can be matched to the context of the available annotations 34 whose context matches the context of the current user.

At an operation 106, the item of the reference content 32 tagged with the annotation context is updated in the database 30 with the annotation 34. In some embodiments, one or more of the annotations 34 to be added to the reference content 32 can be recommended based on the context of the reference content 32.

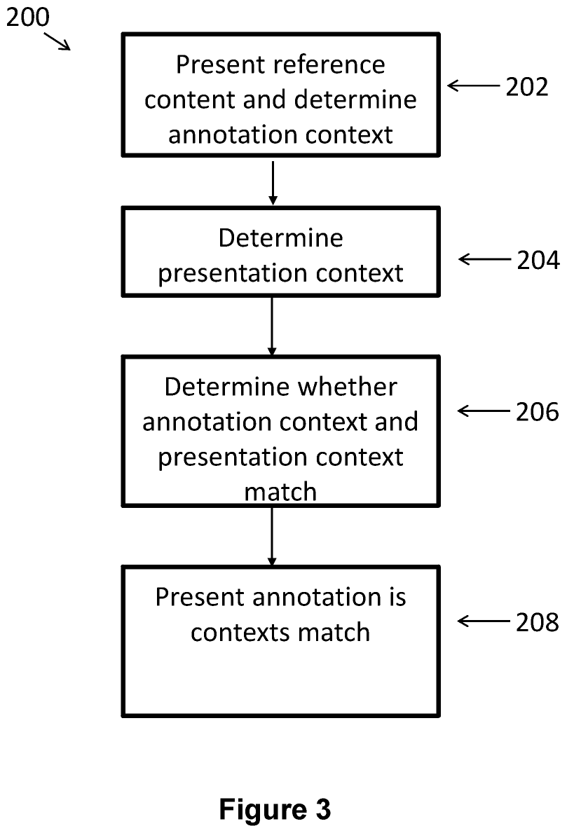

With reference to FIG. 3, and with continuing reference to FIGS. 1 and 2, an illustrative embodiment of an instance of the reference content presentation method 200 is diagrammatically shown as a flowchart. At an operation 202, the item of the reference content 32 (tagged with the annotation 34) is presented via the GUI 28. At an operation 204, a presentation context of the presented reference content 32 is determined. At an operation 206, a determination is made as to whether a comparison of the annotation context and the presentation context satisfies the sharing filter 36. At an operation 208, in response to the sharing filter 36 being satisfied, the annotation 34 of the reference content 32 is presented via the GUI 28. The annotation 34 is not presented if the sharing filter 36 is not satisfied.

### Example

The following provides another example of the educational content monitoring system or apparatus 10. The content management system 10 and the GUI 28 must allow for the user to access content and select it for sharing. When a user has used a piece of content, the system 10 must determine which part of the used content is used to solve a particular problem. The annotation function allows the user to describe why the content helped them and possibly mark parts of the content areas for the attention to the shared community.

The server computer 16 can also include a context engine configured to determine which logged activity patterns and case data related to the content being shared. The context engine examines the context behind the sharing action. This includes the activity, the problem to be solved within the activity and the resolution to the activity. The context engine also records unresolved problems that certain users have experienced, so that it can be used to route future solutions to that particular sub-group of users.

A sharing function dynamically creates sharing options to the selected user groups. This includes “share to all;” “share only to others that have a similar problem to me;” “share only to others in my team.” These levels impact the sensitivity and relevance of the content, and hence serve as a component of the annotation context. Additionally, any personally identifying information (PII) may be removed from the annotation prior to storing it in the database 30. Thus, the system automatically de-identifies, obfuscates or removes parts of the content and annotations depending on how public the sharing level is.

A sharing filter engine and content router can be provided to broadcast to the recipient groups taking those that have recently performed a similar activity/case or have a similar one planned as a priority. The content is cached for sharing and is routed to users to the electronic processing device 18 via a method that prioritizes the content to users that have either experienced the same problem/context in the past and/or routes to users that have planned activities that match the context of the shared resolution. When the shared content is consumed, the recipient of the shared content can indicate whether it helped to solve their problem. If so, the content is prioritized for future content recommendations in for other users with the same access privileges.

While a user is consuming a content item, the user has options to annotate and share the content. When the user deems the content worthy of annotating, she annotates part of the content related to a particular procedure. The user may select the “Share With” icon and selects “Share with others in a similar role”. This will trigger the server computer 14 to determine which logged activity pattern and case that content applies to, and which other users are associated with those activities. This could also include those users who encountered the problems before but were unable to resolve it. This information can be stored in the server computer 16, along with unresolved service action tickets, feedback of the method of solving the problem, and so forth.

The server computer 14 distributes it to those associated users and/or other users who have planned case (e.g. via a scheduling system). Depending on the context of the other users e.g. whether they are internal employees or external contractors, or the level of sharing, the sharing filter may automatically de-identify, obfuscate or remove parts of the content and annotations. This may be tailored specifically to each user, without any intervention from the one who shares.

Users that match such criteria receive the annotated content in their daily clinical content newsfeed. When the shared content is consumed, the recipient of the shared content can indicate whether it helped to solve their problem. If so, the content is prioritized for future content recommendations for other users with the same access privileges.

The function “share with others in a similar role” may be extended to include a predictive capability, namely, to predict users who are highly probable to encounter the similar problems, and include those users in the narrow cast group.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
at least one database storing reference content;
instructions readable and executable by at least one electronic processor to perform an annotation method comprising:
receiving an annotation to an item of the reference content via a user interface (UI);
determining an annotation context of the annotation at least based on analysis of content of the annotation; and
updating the item of the reference content in the database with the annotation tagged with the annotation context.

2. The non-transitory computer readable medium of claim 1, wherein the instructions further include instructions readable and executable by the at least one electronic processor to perform a reference content presentation method comprising:
presenting the item of the reference content via the UI;
determining a presentation context of the presenting;
determining whether a comparison of the annotation context and the presentation context satisfies a sharing filter; and
in response to the sharing filter being satisfied, presenting the annotation via the UI, wherein the annotation is not presented if the sharing filter is not satisfied.

3. The non-transitory computer readable medium of claim 1, wherein the annotation context is determined further based on analyzing additional data of contextual information.

4. The non-transitory computer readable medium of claim 3, wherein the additional data of contextual information includes one or more of:
a location of an electronic processing device used for the receiving of the annotation matched in space and concurrent in time to a location where a medical procedure is being performed,
matching the annotation to an open or recently closed ticket of a medical device maintenance ticketing system, and
information obtainable by matching the annotation to a specific patient.

5. The non-transitory computer readable medium of claim 3, wherein the annotation method further includes:
receiving one or more inputs indicative of a scope of sharing of the annotation;
wherein the additional data of contextual information for determining the annotation context includes the received scope of sharing.

6. The non-transitory computer readable medium of claim 1, wherein the annotation method further includes:
removing personally identifiable information (PII) of persons referenced in the annotation;
wherein the item of the reference content in the database is updated with the annotation after the removal of PII.

7. The non-transitory computer readable medium of claim 1, wherein the reference content comprises one or more of articles, tutorials, or checklists, for use by clinicians when performing medical procedures using a medical device.

8. The non-transitory computer readable medium of claim 1, wherein the annotation includes one or more of text, links to medical images, links to other content, or links to external documentation.

9. A method of presenting reference content stored in a database, the database further storing annotations to the reference content tagged with annotation contexts, the method comprising:
presenting reference content from the database to a current user via a user interface (UI);
matching a context of the current user to the annotation contexts of the stored annotations; and
presenting each annotation whose annotation context matches the context of the current user while not presenting any annotation whose annotation context does not match the context of the current user.

10. The method of claim 9, further comprising:
performing an annotation method including receiving annotations, determining annotation contexts of the received annotations, and storing the annotations tagged with the annotation contexts in the database.

11. The method of claim 9, wherein the annotation context is determined further based on analyzing additional data of contextual information.

12. The method of claim 11, wherein the additional data of contextual information includes one or more of:
a location of an electronic processing device used for the receiving of the annotation matched in space and concurrent in time to a location where a medical procedure is being performed,
matching the annotation to an open or recently closed ticket of a medical device maintenance ticketing system, and
information obtainable by matching the annotation to a specific patient.

13. The method of claim 11, wherein the annotation method further includes:
receiving one or more inputs indicative of a scope of sharing of the annotation;
wherein the additional data of contextual information for determining the annotation context includes the received scope of sharing.

14. The method of claim 10, wherein the annotation method further includes:
removing personally identifiable information (PII) of persons referenced in the annotation;
wherein the item of the reference content in the database is updated with the annotation after the removal of PII.

15. The method of claim 9, wherein the reference content comprises one or more of articles, tutorials, or checklists, for use by clinicians when performing medical procedures using a medical device.

16. The method of claim 9, wherein the annotation includes one or more of text, links to medical images, links to other content, or links to external documentation.

17. A non-transitory computer readable medium storing:
at least one database storing reference content;
instructions readable and executable by at least one electronic processor to perform an annotation method comprising:
receiving an annotation to an item of the reference content via a user interface (UI);
determining an annotation context of the annotation at least based on analysis of content of the annotation; and
updating the item of the reference content in the database with the annotation tagged with the annotation context; and
instructions readable and executable by the at least one electronic processor to perform a reference content presentation method comprising:
presenting the item of the reference content via the UI;
determining a presentation context of the presenting;
determining whether a comparison of the annotation context and the presentation context satisfies a sharing filter; and
in response to the sharing filter being satisfied, presenting the annotation via the UI, wherein the annotation is not presented if the sharing filter is not satisfied.

18. The non-transitory computer readable medium of claim 17, wherein the annotation context is determined further based on analyzing additional data of contextual information.

19. The non-transitory computer readable medium of claim 18, wherein the additional data of contextual information includes one or more of:
a location of an electronic processing device used for the receiving of the annotation matched in space and concurrent in time to a location where a medical procedure is being performed,
matching the annotation to an open or recently closed ticket of a medical device maintenance ticketing system, and
information obtainable by matching the annotation to a specific patient.

20. The non-transitory computer readable medium of claim 18, wherein the annotation method further includes:
receiving one or more inputs indicative of a scope of sharing of the annotation;
wherein the additional data of contextual information for determining the annotation context includes the received scope of sharing.
